(19) United States
(12) Patent Application Publication
(10) Pub. No.: US 2010/0087365 A1
(43) Pub. Date: Apr. 8, 2010

(54) Pharmaceutical Compositions of Hglp-1, Exendin-4 and Analogs Thereof

(76) Inventors: Roland Cherif-Cheikh, Barcelona (ES); Zheng Xin Dong, Holliston, MA (US); Maria Dolores Tobalina Maestre, Barcelona (ES); Jose-Antonio Cordero-Rigol, Barcelona (ES); Frederic Lacombe, Sant Cugat del Valles (ES)

Correspondence Address: Leon R Yankwich;Yankwich & Associates, 201 Broadway, Cambridge, MA, 02139, US

(21) Appl. No.: 12/226,257

(22) PCT Filed: Apr. 13, 2007
(86) PCT No.: PCT/US07/09292

§ 371 Date: Dec. 21, 2009

Related U.S. Application Data
(60) Provisional application No. 60/791,701, filed on Apr. 13, 2006.

(51) Int. Cl. A61K 38/16; A61P 25/00; A61P 3/10

(52) U.S. Cl. 514/12

(57) ABSTRACT

The present invention is directed to pharmaceutical composition comprising a clear solution or an aqueous mixture, a suspension or a semisolid of at least one peptide compound selected from the group consisting of hGLP-1(7-36)-NH₂ and analogs and derivatives thereof, hGLP-1(7-37)-OH and analogs and derivatives thereof and/or exendin-4 and analogs and derivatives thereof, zinc and solvent wherein at least 95% of the said peptide compound is dissolved by the solvent.

This application claims priority to U.S. provisional application No. 60/791,701, filed Apr. 13, 2006.

### BACKGROUND OF THE INVENTION

The present invention is directed to pharmaceutical compositions comprising either human glucagon-like peptide-1 or exendin-4 and/or analogs and derivatives of either hGLP-1 or exedin-4 and to methods of using such pharmaceutical compositions to treat select diseases and/or conditions in humans.

Natural or human synthetic GLP-1 and derivatives thereof are metabolically unstable, having a plasma half life of only one to two minutes in vivo. Once administrated in vivo is also rapidly degraded. This metabolic instability'limits the therapeutic GLP-1. Hence there is a need for specific pharmaceutical composition providing sustained release profile.

The objective of the present invention is to design and provide a formulation able to maintain the biological activity over a prolonged period of time, thanks to the formation of depot at the injection site just after administration.

Additionally, the PK profile obtained from this depot should be as flat as possible taking into account the narrow therapeutic windows of the peptide.

The present invention encompasses pharmaceutical compositions which provide a release of one day up to more than one week.

The pharmaceutical compositions of the present invention could be clear solutions, aqueous suspension or aqueous mixture suspension of solutions, or semi-solid.

Glucagon-like peptide-1 (7-36) amide (GLP-1(7-36)-NH₂) is synthesized in the intestinal L-cells by tissue-specific post-translational processing of the glucagon precursor preproglucagon (Vamdell, J. M., et al., J. Histochem Cytochem, 1985: 33:1080-6) and is released into the circulation in response to a meal. The plasma concentration of GLP-1 rises from a fasting level of approximately 15 μmol/L to a peak postprandial level of 40 μmol/L. It has been denionstrated that, for a given rise in plasma glucose concentration, the increase in plasma insulin is approximately threefold greater when glucose is administered orally compared with intravenously (Kreymann, B., et al., Lancet 1987: 2, 1300-4). This alimentary enhancement of insulin release, known as the incretin effect, is primarily humoral and GLP-1 is now thought to be the most potent physiological incretin in humans. In addition to the insulinotropic effect, GLP-1 suppresses glucagon secretion, delays gastric emptying (Wettergren A., et al., Dig Dis Sci 1993: 38:665-73) and may enhance peripheral glucose disposal (D'Alessio, D. A. et al., J. Clin Invest 1994: 93:2293-6).

In 1994, the therapeutic potential of GLP-1 was suggested following the observation that a single subcutaneous (s/c) dose of GLP-1 could completely normalize postprandial glucose levels in patients with non-insulin-dependent diabetes mellitus (NIDDM) (Gutniak, M. K., et al., Diabetes Care 1994: 17:1039-44). This effect was thought to be mediated both by increased insulin release and by a reduction in glucagon secretion. Furthermore, an intravenous infusion of GLP-1 has been shown to delay postprandial gastric emptying in patients with NIDDM (Williams, B., et al., J. Clin Endo Metab 1996: 81:327-32). Unlike sulphonylureas, the insulinotropic action of GLP-1 is dependent on plasma glucose concentration (Holz, G. G. 4^{th}, et al., Nature 1993: 361:362-5). Thus, the loss of GLP-1-mediated insulin release at low plasma glucose concentration protects against severe hypoglycemia. This combination of actions gives GLP-1 unique potential therapeutic advantages over other agents currently used to treat NIDDM.

Numerous studies have shown that when given to healthy subjects, GLP-1 potently influences glycemic levels as well as insulin and glucagon concentrations (Orskov, C, Diabetologia 35:701-711, 1992; Hoist, J. J., et al, Potential of GLP-1 in diabetes management in Glucagon III, Handbook of Experimental Pharmacology, Lefevbre P J, Ed. Berlin, Springer Verlag, 1996, p. 311-326), effects which are glucose dependent (Kreymann, B., et al., Lancet ii: 1300-1304, 1987; Weir, G. C., et al., Diabetes 38:338-342, 1989). Moreover, it is also effective in patients with diabetes (Gutniak, M., N. Engl J Med 226:1316-1322, 1992; Nathan, D. M., et al., Diabetes Care 15:270-276, 1992), normalizing blood glucose levels in type 2 diabetic subjects (Nauck, M. A., et al., Diagbetologia 36:741-744, 1993), and improving glycemic control in type 1 patients (Creutzfeldt, W. O., et al., Diabetes Care 19:580-586, 1996), raising the possibility of its use as a therapeutic agent.

GLP-1 is, however, metabolically unstable, having a plasma half-life (t_{1/2}) of only 1-2 min in vivo. Exogenously administered GLP-1 is also rapidly degraded (Deacon, C. F., et al., Diabetes 44:1126-1131, 1995). This metabolic instability limits the therapeutic potential of native GLP-1.

A number of attempts have been taken to improve the therapeutic potential of GLP-1 and its analogs through improvements in formulation. For example, International patent publication no. WO 01/57084 describes a process for producing crystals of GLP-1 analogues which are said to be useful in the preparation of pharmaceutical compositions, such as injectable drugs, comprising the crystals and a pharmaceutical acceptable carrier. Heterogeneous micro crystalline clusters of GLP-1(7-37)-OH have been grown from saline solutions and examined after crystal soaking treatment with zinc and/or m-cresol (Kim and Haren, Pharma. Res. Vol. 12 No. 11 (1995)). Crude crystalline suspensions of GLP(7-36)-NH₂ containing needle-like crystals and amorphous precipitation have been prepared from phosphate solutions containing zinc or protamine (Pridal, et. al., International Journal of Pharmaceutics Vol. 136, pp. 53-59 (1996)). European patent publication no. EP 0619322A2 describes the preparation of micro-crystalline forms of GLP-1(7-37)-OH by mixing solutions of the protein in pH 7-8.5 buffer with certain combinations of salts and low molecular weight polyethylene glycols (PEG). U.S. Pat. No. 6,566,490 describes seeding microcrystals of, inter alis, GLP-1 which are said to aid in the production of purified peptide products. U.S. Pat. No. 6,555,521 (US '521) discloses GLP-1 crystals having a tetragonal flat rod or a plate-like shape which are said to have improved purity and to exhibit extended in vivo activity. US '521 teaches that such crystals are relatively uniform and remain in suspension for a longer period of time than prior crystalline clusters and amorphous crystalline suspensions which were said to settle rapidly, aggregate or clump together, clog syringe needles and generally exacerbate unpredictable dosing.

A biodegradable triblock copolymer of poly [(di-lactide-co-glycolide)-b-ethylene glycol-b-(-lactide-co-glycolide)] has been suggested for use in a controlled release formulation of GLP-1. However like other polymeric systems, the manufacture of triblock copolymer involves complex protocols and inconsistent particulate formation.

Similarly, biodegradable polymers, e.g., poly(lactic-co-glycolic acid) (PLGA), have also been suggested for use in sustained delivery formulations of peptides. However the use of such biodegradable polymers has been disfavored in the art since these polymers generally have poor solubility in water and require water-immiscible organic solvents, e.g., methylene chloride, and/or harsh preparation conditions during manufacture. Such organic solvents and/or harsh preparation conditions are considered to increase the risk of inducing conformational change of the peptide or protein of interest, resulting in decreased structural integrity and compromised biological activity. (Choi et al., Pharm. Research, Vol. 21, No. 5, (2004).) Poloxamers have been likewise faulted. (Id.)

The GLP-1 compositions described in the foregoing references are less than ideal for preparing pharmaceutical formulations of GLP's since they tend to trap impurities and/or are otherwise difficult to reproducibly manufacture and administer. Also, GLP analogs are known to induce nausea at elevated concentrations, thus there is a need to provide a sustained drug effect with reduced initial plasma concentrations. Hence, there is a need for GLP-1 formulations which are more easily and reliably manufactured, that are more easily and reproducibly administered to a patient, and that provide for reduced initial plasma concentrations in order to reduce or eliminate unwanted side-effects.

### SUMMARY OF THE INVENTION

The invention may be summarized in the following paragraphs (1) through (28), below, as well as the claims. Accordingly:

(I) In one aspect the present invention is directed to a pharmaceutical composition comprising a clear solution of (a) at least one peptide compound having an aqueous solubility greater than 1 mg/mL at room temperature and a neutral pH which is selected from the group consisting of hGLP-1(7-36)-NH₂ and analogs and derivatives thereof, hGLP-1(7-37)-OH and analogs and derivatives thereof, exendin-4 and analogs and derivatives thereof,

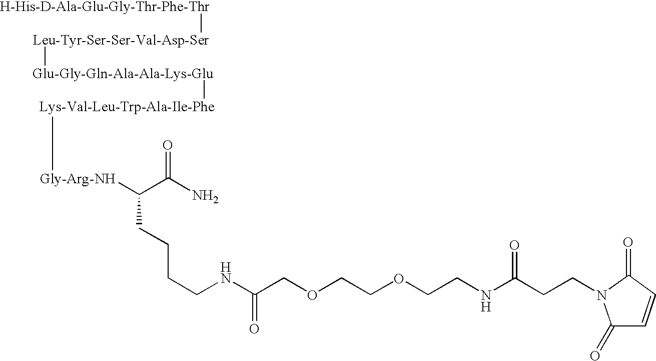

and analogs and derivatives thereof,

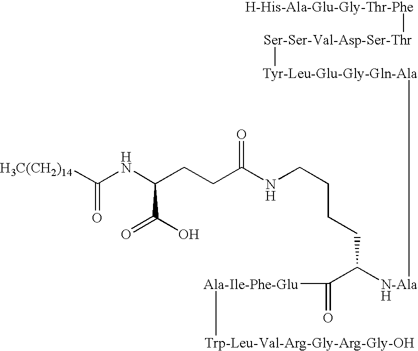

and analogs and derivatives thereof and H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-Lys-Lys-Lys-Lys-Lys-Lys-NH₂ and analogs and derivatives thereof;
(b) a divalent metal ion; and
(c) a solvent
provided that at least 95% of the said peptide compound is dissolved by said solvent.
1. A composition according to paragraphs (I) wherein said divalent metal ion is zinc.
2. In one embodiment the invention features a composition according to paragraphs (I) and (1) wherein said solvent is water.
3. A composition according to paragraph (I) comprising a non-aqueous medium.
4. A composition according to any one of paragraphs (I) to (3), wherein said peptide compound is present in a concentration of about 0.00001-500 mg/mL, preferable about 0.0001-10 mg/mL.
5. A composition according to paragraph (1) wherein said zinc is present in a concentration from 0.0005 mg/mL to 50 mg/mL.
6. A composition according to any one of paragraphs (I) to (5) further comprising a preservative.
7. A composition according to paragraph (6), wherein said preservative is selected from the group consisting of m-cresol, phenol, benzyl alcohol and methyl paraben.
8. A composition according to paragraph (7), wherein said preservative is present in a concentration from 0.01 mg/mL to 50 mg/mL.
9. A composition according to any one of paragraphs (I) to (8) further comprising an isotonic agent.
10. A composition according to paragraphs (I) to (9) wherein said isotonic agent is present in a concentration from 0.01 mg/mL to 50 mg/mL.
11. A composition according to any one of paragraphs (I) to (10) further comprising a stabilizer.
12. A composition according to paragraph (11) wherein said stabilizer is selected from the group consisting of imidazole, arginine and histidine.
13. A composition according to any one of paragraphs (1) to (12) further comprising a surfactant.
14. A composition according to any one of paragraphs (1) to (13) further comprising a chelating agent.
15. A composition according to any one of paragraphs (1) to (14) further comprising a buffer.
16. A composition according to paragraph (15) wherein said buffer is selected from the group consisting of Tris, ammonium acetate, sodium acetate, glycine, aspartic acid and Bis-Tris.
17. A composition according to any one of paragraphs (1) to (16) further comprising a basic polypeptide.
18. A composition according to paragraph (17) wherein said basic polypeptide is selected from the group consisting of polylysine, polyarginine, polyornithine, protamine, putrescine, spermine, spermidine and histone.
19. A composition according to any one of paragraphs (1) to (18) further comprising alcohol or a mono- or di-saccharide.
20. A composition according to paragraph (19) wherein said alcohol or mono- or di-saccharide is selected from the group consisting of methanol, ethanol, propanol, glycerol, trehalose, mannitol, glucose, erythrose, ribose, galactose, fructose, maltose, sucrose and lactose.
21. A composition according to any one of paragraphs (1) to (20) further comprising ammonium sulfate.
22. A pharmaceutical composition comprising an effective amount of a compound according to paragraphs (1) through (21) or a pharmaceutically acceptable salt thereof and a pharmaceutically acceptable carrier or diluent.
23. A method of eliciting an agonist effect from a GLP-1 receptor in a subject in need thereof, which comprises administering to said subject an effective amount of a compound according to paragraph (1) or paragraph (22) or a pharmaceutically acceptable salt thereof.
24. A method of treating a disease selected from the group consisting of Type I diabetes, Type II diabetes, obesity, glucagonomas, secretory disorders of the airway, metabolic disorder, arthritis, osteoporosis, central nervous system disease, restenosis and neurodegenerative disease, in a subject in need thereof, which comprises administering to said subject an effective amount of a composition according to paragraph (1) or a pharmaceutically acceptable salt thereof.
25. In yet another aspect, the present invention provides a method of eliciting an agonist effect from a GLP-1 receptor in a subject in need thereof which comprises administering to said subject a formulation of the instant present invention comprising an effective amount of a compound of paragraph (I) as defined hereinabove or a pharmaceutically acceptable salt thereof.
26. In a further aspect, the present invention provides a method of treating a disease selected from the group consisting of Type I diabetes, Type II diabetes, obesity, glucagonomas, secretory disorders of the airway, metabolic disorders, arthritis, osteoporosis, central nervous system disease, restenosis, neurodegenerative disease, renal failure, congestive heart failure, nephrotic syndrome, cirrhosis, pulmonary edema, hypertension and disorders wherein the reduction of food intake is desired, in a subject in need thereof, which comprises administering to said subject for use in a formulation of the present invention comprising an effective amount of a compound of paragraph (I) as defined hereinabove or a pharmaceutically acceptable salt thereof.
27. A preferred method of paragraph (26) is where the disease being treated is Type I diabetes or Type II diabetes.

(II). In a second aspect the present invention is directed to pharmaceutical composition comprising a clear solution or an aqueous mixture, a suspension or a semisolid pharmaceutical composition of (a) at least one peptide compound having an aqueous solubility greater than 1 mg/mL at room temperature and having a pH from 3.0 to 8.0, and preferably a pH from 4.0 to 6.0 which is selected from the group consisting of hGLP-1(7-36)-NH₂ and analogs and derivatives thereof, hGLP-1(7-37)-OH and analogs and derivatives thereof, exendin-4 and analogs and derivatives thereof,

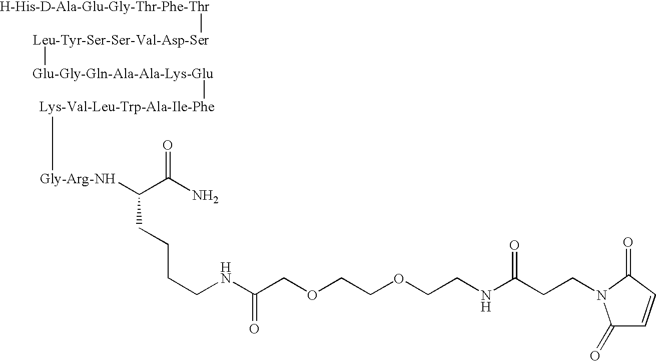

and analogs and derivatives thereof,

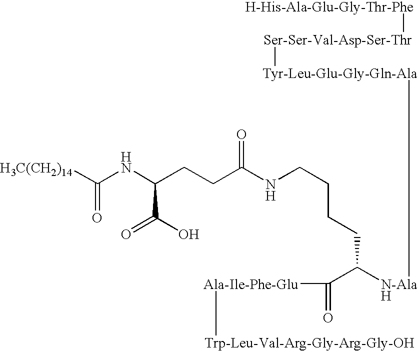

and analogs and derivatives thereof and H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-Lys-Lys-Lys-Lys-Lys-Lys-NH₂ and analogs and derivatives thereof;
(b) a divalent metal ion; and
(c) a solvent
provided that less than 95%±5% of the said peptide compound is dissolved by said solvent. The reference number of the second aspect of the invention 1 to 27 are the number under paragraph II.
1. A composition according to paragraphs (II) wherein said divalent metal ion is zinc.
2. In one embodiment the invention features a composition according to paragraphs (II) and (1) wherein said solvent is water.
3. A composition according to paragraph (II) comprising a non-aqueous medium.
4. A composition according to any one of paragraphs (11) to (3), wherein said peptide compound is present in a concentration of about 0.00001-500 mg/mL or 0.00001-500 mg/g, preferable about 50-350 mg/ml or 50-350 mg/g
5. A composition according to paragraph (1) wherein said zinc is present in a concentration from 0.0005 mg/mL to 50 mg/mL.
6. A composition according to any one of paragraphs (II) to (5) further comprising a preservative.
7. A composition according to paragraph (6), wherein said preservative is selected from the group consisting of m-cresol, phenol, benzyl alcohol and methyl paraben.
8. A composition according to paragraph (7), wherein said preservative is present in a concentration from 0.01 mg/mL to 50 mg/mL.
9. A composition according to any one of paragraphs (II) to (8) further comprising an isotonic agent.
10. A composition according to paragraphs (II) to (9) wherein said isotonic agent is present in a concentration from 0.01 mg/mL to 50 mg/mL.
11. A composition according to any one of paragraphs (II) to (10) further comprising a stabilizer.
12. A composition according to paragraph (11) wherein said stabilizer is selected from the group consisting of imidazole, arginine and histidine.
13. A composition according to any one of paragraphs (II) to (12) further comprising a surfactant.
14. A composition according to any one of paragraphs (II) to (13) further comprising a chelating agent.
15. A composition according to any one of paragraphs (II) to (14) further comprising a buffer.
16. A composition according to paragraph (15) wherein said buffer is selected from the group consisting of Tris, ammonium acetate, sodium acetate, glycine, aspartic acid and Bis-Tris.
17. A composition according to any one of paragraphs (II) to (16) further comprising a basic polypeptide.
18. A composition according to paragraph (17) wherein said basic polypeptide is selected from the group consisting of polylysine, polyarginine, polyornithine, protamine, putrescine, spermine, spermidine and histone.
19. A composition according to any one of paragraphs (II) to (18) further comprising alcohol or a mono- or di-saccharide.
20. A composition according to paragraph (19) wherein said alcohol or mono- or di-saccharide is selected from the group consisting of methanol, ethanol, propanol, glycerol, trehalose, mannitol, glucose; erythrose, ribose, galactose, fructose, maltose, sucrose and lactose.
21. A composition according to any one of paragraphs (II) to (20) further comprising ammonium sulfate.
22. A pharmaceutical composition comprising an effective amount of a compound according to paragraphs (II) through (21) or a pharmaceutically acceptable salt thereof and a pharmaceutically acceptable carrier or diluent.
23. A method of eliciting an agonist effect from a GLP-1 receptor in a subject in need thereof, which comprises administering to said subject an effective amount of a compound according to paragraph (II) or paragraph (23) or a pharmaceutically acceptable salt thereof.
24. A method of treating a disease selected from the group consisting of Type I diabetes, Type II diabetes, obesity, glucagonomas, secretory disorders of the airway, metabolic disorder, arthritis, osteoporosis, central nervous system disease, restenosis and neurodegenerative disease, in a subject in need thereof, which comprises administering to said subject an effective amount of a composition according to paragraph (II) or a pharmaceutically acceptable salt thereof.
25. In yet another aspect, the present invention provides a method of eliciting an agonist effect from a GLP-1 receptor in a subject in need thereof which comprises administering to said subject a formulation of the instant present invention comprising an effective amount of a compound of paragraph (29) as defined hereinabove or a pharmaceutically acceptable salt thereof.
26. In a further aspect, the present invention provides a method of treating a disease selected from the group consisting of Type I diabetes, Type II diabetes, obesity, glucagonomas, secretory disorders of the airway, metabolic disorders, arthritis, osteoporosis, central nervous system disease, restenosis, neurodegenerative disease, renal failure, congestive heart failure, nephrotic syndrome, cirrhosis, pulmonary edema, hypertension and disorders wherein the reduction of food intake is desired, in a subject in need thereof, which comprises administering to said subject for use in a formulation of the present invention comprising an effective amount of a compound of paragraph (II) as defined hereinabove or a pharmaceutically acceptable salt thereof.
27. A preferred method of paragraph (26) is where the disease being treated is Type I diabetes or Type II diabetes.

### BRIEF DESCRIPTION OF THE DRAWINGS

FIG. 1 shows a peptide plasma profile obtained after single sc administration to dogs of aqueous composition 100 mg/g hGLP-1(7-36)-NH₂ with Zn, at D=15 mg peptide.

All abbreviations (e.g. Ala) of amino acids in this disclosure stand for the structure of —NH—CR′R²—CO—, wherein R¹ and R² are the side chains of an amino acid (e.g., R¹═CH₃ and R²═H for Ala). Amp, 1-NaI, 2-NaI, Nle, Cha, 3-Pal, 4-Pal and Aib are the abbreviations of the following α-amino acids: 4-amino-phenylalanine, β-(1-naphthyl)alanine, β-(2-naphthyl)alanine, norleucine, cyclohexylalanine, β-(3-pyridinyl)alanine, β-(4-pyridinyl)alanine and α-aminoisobutyric acid, respectively. Other amino acid definitions are: Ura is urocanic acid; Pta is (4-pyridylthio) acetic acid; Paa is trans-3-(3-pyridyl) acrylic acid; Tma-His is N,N-tetramethylamidino-histidine; N-Me-Ala is N-methyl-alanine; N-Me-Gly is N-methyl-glycine; N-Me-Glu is N-methyl-glutamic acid; Tle is tert-butylglycine; Abu is α-aminobutyric acid; Tba is tert-butylalanine; Orn is ornithine; Aib is α-aminoisobutyric acid; β-Ala is β-alanine; Gaba is γ-aminobutyric acid; Ava is 5-aminovaleric acid; Ado is 12-aminododecanoic acid, Aic is 2-aminoindane-2-carboxylic acid; Aun is 11-aminoundecanoic acid; and Aec is 4-(2-aminoethyl)-1-carboxymethyl-piperazine, represented by the structure:

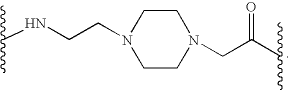

What is meant by Acc is an amino acid selected from the group of 1-amino-1-cyclopropanecarboxylic acid (A3c); 1-amino-1-cyclobutanecarboxylic acid (A4c); 1-amino-1-cyclopentanecarboxylic acid (A5c); 1-amino-1-cyclohexanecarboxylic acid (A6c); 1-amino-1-cycloheptanecarboxylic acid (A7c); 1-amino-1-cyclooctanecarboxylic acid (A8c); and 1-amino-1-cyclononanecarboxylic acid (A9c). In the above formula, hydroxyalkyl, hydroxyphenylalkyl, and hydroxynaphthylalkyl may contain 1-4 hydroxy substituents. COX⁵ stands for —C═O.X⁵. Examples of —C═O.X⁵ include, but are not limited to, acetyl and phenylpropionyl.

The full names for other abbreviations used herein are as follows: Boc for t-butyloxycarbonyl, HF for hydrogen fluoride, Fm for formyl, Xan for xanthyl, Bzl for benzyl, Tos for tosyl, DNP for 2,4-dinitrophenyl, DMF for dimethylformamide, DCM for dichloromethane, HBTU for 2-(1H-Benzotriazol-1-yl)-1,1,3,3-tetramethyl uronium hexafluorophosphate, DIEA for diisopropylethylamine, HOAc for acetic acid, TFA for trifluoroacetic acid, 2CIZ for 2-chlorobenzyloxycarbonyl, 2BrZ for 2-bromobenzyloxycarbonyl, OcHex for O-cyclohexyl, Fmoc for 9-fluorenylmethoxycarbonyl, HOBt for N-hydroxybenzotriazole; PAM resin for 4-hydroxymethylphenylacetamidomethyl resin; Tris for Tris(hydroxymethyl)aminomethane; and Bis-Tris for Bis(2-hydroxyethyl)amino-tris(hydroxymethyl)methane (i.e., 2-Bis(2-hydroxyethyl)amino-2-(hydroxymethyl)-1,3-propanediol).

The term “halo” or “halogen” encompasses fluoro, chloro, bromo and iodo.

The terms “(C₁-C₁₂)hydrocarbon moiety”, “(C₁-C₃₀)hydrocarbon moiety” and the like encompass branched and straight chain alkyl, alkenyl and alkynyl groups having the indicated number of carbons, provided that in the case of alkenyl and alkynyl there is a minimum of two carbons.

A peptide of this invention is also denoted herein by another format, e.g., (A5c⁸)ₕGLP-1(7-36)NH₂, with the substituted amino acids from the natural sequence placed between the first set of parentheses (e.g., A5c⁸ for Ala⁸ in hGLP-1). The abbreviation GLP-1 means glucagon-like peptide-1; hGLP-1 means human glucagon-like peptide-1. The numbers between the parentheses refer to the number of amino acids present in the peptide (e.g., hGLP-1(7-36) is amino acids 7 through 36 of the peptide sequence for human GLP-1). The sequence for hGLP-1(7-37) is listed in Mojsov, S., Int. J. Peptide Protein Res, 40, 1992, pp. 333-342. The designation “NH₂” in hGLP-1(7-36)NH₂ indicates that the C-terminus of the peptide is amidated. hGLP-1(7-36) means that the C-terminus is the free acid. In hGLP-1(7-38), residues in positions 37 and 38 are Gly and Arg, respectively, unless otherwise indicated. The sequence for exendin-4 is listed in J. W. Neidigh, et al. Biochemistry, 2001, 40, pp 13188-13200.

What is meant by a “clear solution” is a solution comprised of a solvent and one or more solutes wherein 95%±5%, preferably 99%, of the solute is completely dissolved so that the solution is relatively transparent. A clear solution may have trace amounts of undissolved, observable solute and/or inactive other particles depending on the purity of the solvent used, however, such particles are not in a sufficient quantity to create a milky or cloudy appearance. A clear solution does not apply to a suspension which is a heterogeneous mixture composed of a diverse and continuous phase, whereas a solution is a homogeneous, single-phase mixture of two or more substances.

What is meant by an aqueous mixture, by a suspension or by semisolid is a formulation comprised of a solvent and one or more solutes wherein the solute may be partially dissolved, so that the formulation is not a transparent composition that could be as liquid as a clear solution or more viscous, depending on solute concentration, but still injectable using fine needles.

The peptides used in this invention advantageously may be provided in the form of pharmaceutically acceptable salts. Examples of such salts include, but are not limited to, those formed with organic acids (e.g., acetic, lactic, maleic, citric, malic, ascorbic, succinic, benzoic, methanesulfonic, toluenesulfonic or pamoic acid, trifluoroacetic acid (TFA)), inorganic acids (e.g., hydrochloric acid, sulfuric acid, or phosphoric acid), and polymeric acids (e.g., tannic acid, carboxymethyl cellulose, polylactic, polyglycolic or copolymers of polylactic-glycolic acids).

A typical method of making a salt of a peptide of the present invention is well known in the art and can be accomplished by standard methods of salt exchange.

As is well known to those skilled in the art, the known and potential uses of GLP-1 are varied and multitudinous (See, Todd, J. F., et al., Clinical Science, 1998, 95, pp. 325-329; and Todd, J. F. et al., European Journal of Clinical Investigation, 1997, 27, pp. 533-536). Thus, the administration of naturally-occurring GLP-1 (i.e., hGLP-1(7-36)-NH₂ and hGLP-1(7-37)-OH), exedin-4, PC-DAC®, Liraglutide® and/or AVE-0010/ZP-10 according to this invention for purposes of eliciting an agonist effect can greatly advance the treatment of various debilitating diseases and conditions known to be treatable by GLP-1 such as: Type I diabetes, Type II diabetes, obesity, glucagonomas, secretory disorders of the airway, metabolic disorder, arthritis, osteoporosis, central nervous system diseases, restenosis, neurodegenerative diseases, renal failure, congestive heart failure, nephrotic syndrome, cirrhosis, pulmonary edema, hypertension, and disorders wherein the reduction of food intake is desired.

Accordingly, the present invention includes within its scope pharmaceutical compositions as defined herein comprising, as an active ingredient, at least one of the compounds of paragraph (I).

The dosage of active ingredient in the formulations of this invention may be varied; however, it is necessary that the amount of the active ingredient be such that a suitable dosage is obtained. The selected dosage depends upon the desired therapeutic effect, on the route of administration, and on the duration of the treatment, and normally will be determined by the attending physician. In general, an effective dosage for the activities of this invention is in the range of 1×10⁻⁷ to 200 mg/kg/day, preferably 1×10⁻⁴ to 100 mg/kg/day, which can be administered as a single dose or divided into multiple doses.

The formulations of this invention are preferably administered parenterally, e.g., intramuscularly, intraperitoneally, intravenously, subcutaneously, and the like.

Preparations according to this invention for parenteral administration include sterile aqueous or non-aqueous solutions, suspensions, gels, or emulsions, provided that the desired in vivo release profile is achieved. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be dissolved in sterile water, or some other sterile injectable medium immediately before use.

Unless defined otherwise, all technical and scientific terms used herein have the same meaning as commonly understood by one of ordinary skill in the art to which this invention belongs. Also, all publications, patent applications, patents and other references mentioned herein are incorporated by reference.

### DETAILED DESCRIPTION

### Synthesis of Peptides

Peptides useful for practicing the present invention can be and were prepared by standard solid phase peptide synthesis. See, e.g., Stewart, J. M., et al., Solid Phase Synthesis (Pierce Chemical Co., 2d ed. 1984). The substituents may be attached to the free amine of the Lys or other amino acid residues by standard methods known in the art. For example, an acyl group may be attached by coupling the free acid to the free amine of a residue by mixing the partially protected peptide-resin with 3 molar equivalents of both the free acid and diisopropylcarbodiimide in methylene chloride for one hour.

hGLP-1(7-36)-NH₂ peptide was synthesized on an Applied Biosystems (Foster City, Calif.) model 430A peptide synthesizer which was modified to do accelerated Boc-chemistry solid phase peptide synthesis. See Schnolzer, et al., Int. J. Peptide Protein Res., 90:180 (1992). 4-methylbenzhydrylamine (MBNA) resin (Peninsula, Belmont, Calif.) was used. The Boc amino acids (Bachem, Calif., Torrance, Calif.; Nova Biochem., LaJolla, Calif.) were used with the following side chain protection: Boc-Ala-OH, Boc-Arg(Tos)-OH, Boc-Asp(OcHex)-OH, Boc-Tyr(2BrZ)-OH, Boc-His(DNP)-OH, Boc-Val-OH, Boc-Leu-OH, Boc-Gly-OH, Boc-Gln-OH, Boc-Ile-OH, Boc-Lys(2CIZ)-OH, Boc-Thr(Bzl)-OH, Boc-Ser(Bzl)-OH, Boc-Phe-OH, Boc-Glu(OcHex)-OH and Boc-Trp(Fm)-OH. The Boc groups were removed by treatment with 100% TFA for 2×1 min. Boc amino acids were pre-activated with HBTU and DIEA in DMF and were coupled without prior neutralization of the peptide-resin TFA salt. Coupling times were 5 min.

At the end of the assembly of the peptide chain, the resin was treated with a solution of 20% mercaptoethanol/10% DIEA in DMF for 2×30 min. The N-terminal Boc group was then removed by treatment with 100% TFA for 2×2 min. After neutralization of the peptide-resin with 10% DIEA in DMF (1×1 min), the formyl group on the side chain of Trp was removed by treatment with a solution of 15% ethanolamine/15% water/70% DMF for 2×30 min. The peptide-resin was washed with DMF and DCM and dried under reduced pressure. The final cleavage was done by stirring the peptide-resin in HF containing anisole and dithiothreitol at 0° C. for 75 min. HF was removed by a flow of nitrogen. The residue was washed with ether and extracted with 4N HOAc.

The peptide mixture in the aqueous extract was purified on reverse-phase preparative high pressure liquid chromatography (HPLC) using a reverse phase VYDAC® C₁₈ column (Nest Group, Southborough, Mass.). The column was eluted with a linear gradient (20% to 50% of solution B over 105 min.) at a flow rate of 10 mL/min (Solution A=water containing 0.1% TFA; Solution B=acetonitrile containing 0.1% of TFA). Fractions were collected and checked on analytical HPLC. Those containing pure product were combined and lyophilized to dryness. Purity of the final peptide was checked on an analytical HPLC system. Electro-spray mass spectrometer (MS (ES))S analysis was used to check the molecular weight of the final product.

The TFA peptide salts of the present invention results from the purification of the peptide by using preparative HPLC, eluting with TFA containing buffer solutions.TFA salts can be converted into another salt, such as an acetate salt by dissolving the peptide in a small amount of 0.25 N acetic acid aqueous solution. The resulting solution is applied to a semi-prep HPLC column (Zorbax, 300 SB, C-8). The column is eluted with (1) 0.1N ammonium acetate aqueous solution for 0.5 hrs., (2) 0.25N acetic acid aqueous solution for 0.5 hrs. and (3) a linear gradient (20% to 100% of solution B over 30 min.) at a flow rate of 4 ml/min (solution A is 0.25N acetic acid aqueous solution; solution B is 0.25N acetic acid in acetonitrile/water, 80:20). The fractions containing the peptide are collected and lyophilized to dryness:

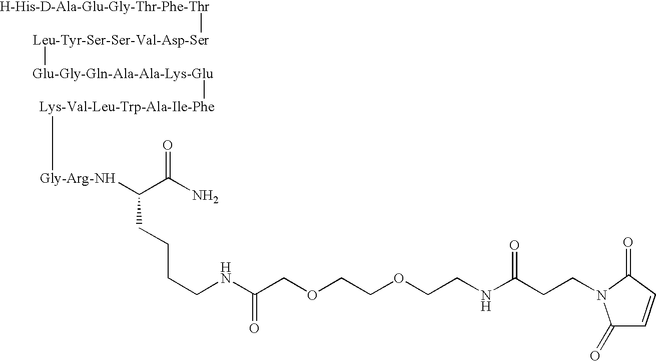

is sold under the trademark PC-DAC® and is the property of Conjuchem, Montreal, Quebec, Canada. Discussed peptide:

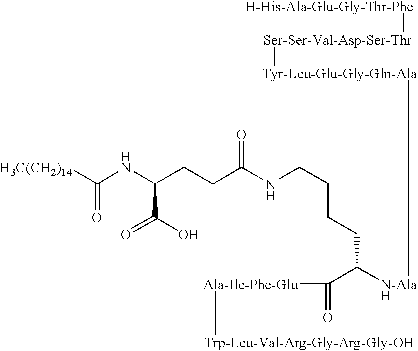

is sold as Liraglutide® and is the property of Novo Nordisk, Bagsvaerd, Denmark. The discussed peptide

H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-Lys-Lys-Lys-Lys-Lys-Lys-NH₂ is referred to in the prior art as “AVE-0010/ZP-10” and is the joint property of Sanofi-Aventis, Paris, France and Zealand Pharma, Glostrup, Denmark.

### EXPERIMENTAL PROCEDURES

### A. Determination of GLP-1 Receptor Affinity

Compounds useful to practice the present invention can be tested for their ability to bind to the GLP-1 receptor using the following procedure.

### Cell Culture:

RIN 5F rat insulinoma cells (ATCC-# CRL-2058, American Type Culture Collection, Manassas, Va.), expressing the GLP-1 receptor, are cultured in Dulbecco's modified Eagle's medium (DMEM) containing 10% fetal calf serum, and are maintained at about 37° C. in a humidified atmosphere of 5% CO₂/95% air.

### Radioligand Binding:

Membranes are prepared for radioligand binding studies by homogenization of the RIN cells in 20 ml of ice-cold 50 mM Tris-HCl with a Brinkman Polytron (Westbury, N.Y.) (setting 6, 15 sec). The homogenates are washed twice by centrifugation (39,000 g/10 min), and the final pellets are re-suspended in 50 mM Tris-HCl, containing 2.5 mM MgCl₂, 0.1 mg/ml bacitracin (Sigma Chemical, St. Louis, Mo.), and 0.1% BSA. For assay, aliquots (0.4 ml) are incubated with 0.05 nM (¹²⁵I)GLP-1(7-36) (˜2200 Ci/mmol, New England Nuclear, Boston, Mass.), with and without 0.05 ml of unlabeled competing test peptides. After a 100 min incubation (25° C.), the bound (¹²⁵I)GLP-1(7-36) are separated from the free by rapid filtration through GF/C filters (Brandel, Gaithersburg, Md.), which are previously soaked in 0.5% polyethyleneimine. The filters are then washed three times with 5 ml aliquots of ice-cold 50 mM Tris-HCl, and the bound radioactivity trapped on the filters is counted by gamma spectrometry (Wallac LKB, Gaithersburg, Md.). Specific binding is defined as the total (¹²⁵I)GLP-1(7-36) bound minus that bound in the presence of 1000 nM GLP1(7-36) (Bachem, Torrence, Calif.).

### B. Determination of Solubility vs pH

Advantageously, compounds for use in the present invention are relatively soluble in aqueous solutions at certain pH and are relatively insoluble in aqueous solutions in the presence of divalent metal ions, such as zinc. Compounds for use in the present invention have an aqueous solubility greater than 1 mg/mL at neutral pH at room temperature.

### Determination of Compound Aqueous Solubility at pH 7:

Compounds that may advantageously be used to practice the invention can be tested to determine their solubility at either room temperature or approximately 37° C. in water using the following procedure.

To determine the solubility at room temperature, 2 mg of hGLP-1(7-36)-NH₂ is weighed and deposited into a glass vial and a 200 uL aliquot of de-ionized water is then added to the vial. The procedure takes place in a room which is maintained at approximately 25° C. The pH of the resulting solution is measured to be approximately 5. The peptide sample dissolves instantly and a clear solution is observed. A neutral pH (pH 7) is achieved by treating the sample solution with a small amount of 0.1 N NaOH. The neutral solution is observed to be clear thus indicating that the solubility of hGLP-1(7-36)-NH₂ is greater than 10 mg/mL at room temperature at neutral pH.

To determine the solubility at 37° C., 2 mg of hGLP-1(7-36)-NH₂ is weighed and deposited into a glass vial and a 200 uL aliquot of de-ionized water is then added to the vial. The procedure takes place in a room which is maintained at approximately 37° C. The pH of the resulting solution is measured to be approximately 5. The peptide sample dissolved instantly and a clear solution is observed. A neutral pH (pH 7) is obtained by treating the sample solution with a small amount of 0.1N NaOH. The neutral solution is observed to be clear thus indicating that the solubility of hGLP-1(7-36)-NH₂ is greater than 10 mg/mL at 37° C.

### C. Determination of Aqueous Solubility of Compound vs Zinc Concentration

Compounds that may advantageously be used to practice the invention can be tested to determine their solubility in pH 7 water at different zinc concentrations using the following procedure.

A stock zinc solution is prepared by dissolving ZnCl₂ in de-ionized water to a concentration of 100 mg/ml and adjusting the pH to 2.7 using HCl. Solutions having various ZnCl₂ concentrations (“Zn Test Solutions”) are prepared by making appropriate dilutions of the stock solution.

A 1 mg sample of the tested compound is dissolved in 250 μl of each tested Zn solution to yield a solution having 4 mg/ml of the tested compound. The pH of this solution is then adjusted using 0.2 N NaOH until white precipitates form. The precipitation solution is centrifuged and the mother liquor is analyzed using HPLC. The UV absorption area of test compound peak is measured and the concentration of the tested compound in the mother liquor is determined via comparison to a calibration curve.

### D. In Vivo Assays

Compositions of the present invention can be and were tested to determine their ability to promote and enhanced effect in vivo using the following assays.

### Experimental Procedure—24 Hours:

The day prior to the experiment, adult male Sprague-Dawley rats (Taconic, Germantown, N.Y.) that weighed approximately 300-350 g are implanted with a right atrial jugular cannula under chlorohydrate anesthetic. The rats are then fasted for 18 hours prior to the injection of the appropriate test composition or vehicle control at time 0. The rats continue to be fasted throughout the entire experiment.

At time zero the rats are injected subcutaneously (sc) either with tested compounds at pH 4.0 or pH 7.0 as a clear solution. In both cases the injection volume is very small (4-6 μL) and the dose of GLP-1 compound administered to the subject is 75 μg/kg. At the appropriate time after the sc injections a 5000 blood sample is withdrawn via the intravenous (iv) cannula and the rats are given an iv glucose challenge to test for the presence of enhanced insulin secretion. The times of the glucose challenge are 0.25, 1, 6, 12 and 24 hours post-compound injection. After the initial blood sample is withdrawn glucose (1 g/kg) is injected iv and flushed in with 500 μl heparinized saline (10 U/mL). Thereafter, 500 μl blood samples are withdrawn at 2.5, 5, 10 and 20 minutes post-glucose injection. Each of these is immediately followed by an iv injection of 500 μl heparinized saline (10 U/mL) through the cannula. The blood samples are centrifuged, plasma is collected from each sample and the samples are stored at ±20° C. until they are assayed for insulin content. The amount of insulin in each sample is determined using a rat insulin enzyme-linked immunosorbant assay (ELISA) kit (American Laboratory Products Co., Windham, N.H.).

### Results:

A sustained insulin-enhancing activity is observed that is inducible by glucose injection over the full 24 hours of the experiment.

### Experimental Procedure—Extended Term:

The general procedure is the same as previously described. In this case, either a tested compound or a vehicle control is injected subcutaneously (“sc”) at time zero. The time points for the glucose challenge are 1, 6, 12, 24, 48 and 72 hours post-injection. The glucose injection via the iv cannula and subsequent blood sampling are performed as in the previously described experiment. Because of the extended fasting period, vehicle and glucose-only controls are included at each time point.

### Results:

A sustained insulin-enhancing activity that is inducible by glucose for at least 48 hours after subcutaneous injection of the tested composition is observed. In addition, as in the previously described experiment, no initial high level of insulin enhancement in response to glucose is observed.

### E. In Vivo Tests

Compositions of the present invention can be and were tested to determine their ability to promote extended release of active compound in vivo using assays E.1-E.4, described below.

Compositions for use in the assays below were made according to the following general procedure:

Stock solutions of 100 mg/ml ZnCl₂ were made by dissolving zinc chloride (Merck, Mollet del Valles, Barcelona, Spain) in sterile water for injection (Braun, Rubi, Spain) which had been adjusted to pH 2.7 using HCl. Solutions containing zinc at various concentrations, e.g., 0.1 mg/ml, 0.5 mg/ml, 2 mg/ml, etc., were obtained by dilution of the stock solution. Solutions containing zinc at lower concentrations, e.g., 10 μg/ml, 20 μg/ml, 30 μg/ml, were prepared in an analogous manner by dilution of a stock solution comprising 1 mg/ml ZnCL₂.

An appropriate amount of a compound to be assayed was weighed and dissolved in the appropriate volume of each resulting zinc solution to yield a clear solution having a desired concentration of the compound; e.g., 4 mg/ml. The resulting solutions were then micro-filtered and, if necessary, stored in light-protected vials before administration.

The concentration of test compound in the plasma of the test subjects may be determined by a number of methods known in the art. In one convenient method the concentration of a compound is determined via radioimmunoassay employing a rabbit derived antibody to the test compound in competition with a known quantity of test compound that has been radio-iodinated with, e.g., ¹²⁵I.

### E.1. Pharmacokinetic Study 1

The effect of zinc on the bioavailability of a bioactive compound administered to a subject using a composition according to the invention can be and was determined as follows.

Following the procedures described above, four aqueous compositions were formulated to have 4 mg/mL of the tested compounds at pH=2.7, and 0.0, 0.1, 0.5, and 2.0 mg/ml of ZnCL₂, respectively. Each of the four compositions was administered subcutaneously to 16 Sprague-Dawley rats (Charles River Laboratories, Wilmington, Mass., USA). The average age of the rats was approximately 8-9 weeks, and the average weight was approximately 260-430 g. The rats were provided food and water ad libitum.

### E.2. Pharmacokinetic Study 2

The effect of injection volume on the bioavailability of a bioactive compound administered to a subject using a composition according to the invention can be and was determined as follows.

Following the procedures described above, three aqueous compositions were formulated to have 3000, 300 and 75 microg/mL, respectively, at a pH of 2.7 and Zn concentration of 0.5 mg/ml. Each of the three compositions was administered subcutaneously to 16 Sprague-Dawley rats (Charles River Laboratories, Wilmington, Mass., USA). The average age of the rats was approximately 8-10 weeks and the average weight was approximately 330-460 g. The rats were fasted overnight prior to commencement of the study. The volume of injection was selected to provide each rat with 75 micorg/kg dose of the tested compound. (0.025 ml/kg, 0.25 ml/kg, and 1 ml/kg, respectively.)

### E.3. Pharmacokinetic Study 3

The effect of zinc on the bioavailability of a bioactive compound administered to a subject using a composition according to the invention can be and was determined as follows.

Following the procedures described above, three aqueous compositions were formulated to have 4 mg/mL of the tested compounds at pH=2.7, and 10, 20 and 30 microg/mL of zinc, respectively. Each of the three compositions was administered subcutaneously to 16 Male albino Sprague-Dawley rats (St. Feliu de Codines, Barcelona, ES). These rats were fasted overnight prior to commencement of the study.

### E.4. Pharmacokinetic Study 4

The effect of zinc and bioactive compound concentrations on the bioavailability of the bioactive compound when administered to a subject using a composition according to the invention can be and was determined as follows.

Following the procedures described above, two aqueous compositions were formulated. The first solution comprised 1.45 mg/ml of the tested compounds and 30 micorg/ml Zinc, the second comprised 1.45 mg/ml of the compound, but without zinc. Both solutions had pH=2.7. Each solution was administered subcutaneously to male Beagle dogs (Isoquimen, Barcelona, Spain) ranging in age from approximately 54-65 months and in weight from approximately 16-21 kg. The dogs were fasted overnight prior to commencement of the study. Additionally, the second solution containing only active compound was administered intravenously.

### E.5. Example of Pharmacokinetic Study

This part discloses the preparation and administration of a composition of 100 mg/g natural human glucagon-like peptide-1, hGLP-1(7-36)-NH₂ peptide aqueous formulation (w/w), with Zn (from a ZnCl₂), being in the molar ratio of [Peptide:Zn]=1.5:1

The substance tested is natural hGLP-1(7-36)-NH₂ and was provided by (Polypeptide, USA).

E.5.1. Preparation Procedure

The peptide compound was weighed and mixed with a weighed amount of ZnCl₂ solution, 1.474 mg Zn/ml, to have a final peptide concentration of 100 mg/g and a final molar ratio [Peptide:Zn]=1.5:1

Syringes with 29G needle (0.33 mm) were filled with the amount of composition required to administer a 15 mg dose of peptide. Upon preparation, the samples were analysed and the composition was administered to male Beagle dogs.

The following analytical results were obtained:

Peptide Content: 10.31+/−0.03% w/w

Injected Dose: 15.71+/−0.18 mg

HPLC Purity: 98.5% Ar
4.5

The molar ratio value for the composition was [Peptide:Zn]=1.44:1

E.5.2. PK Study, Bioanalysis and Results

The aim of this study was to assess the serum pharmacokinetic profile of the natural hGLP-1(7-36)-NH₂ following single subcutaneous administration to male Beagle dogs of a formulation of 100 mg/g GLP-1(7-36)-NH₂ acetate with ZnCl₂, molar ratio [peptide:Zn]=1.5:1, at a total theoretical dose of 15 mg of pure peptide.

The composition was administered the day of preparation at a theoretical dose of 15 mg of pure peptide (aprox 150 μl) to male Beagle dogs.

A total of 6 male Beagle dogs, 33 to 84 months old and 12 to 25 kg bodyweight were used. They were maintained with free access to a dry standard diet and to drinkable water, both were checked periodically.

The animals were fasted 6 h more than usual (about 18 h of fasted period before administration) to avoid a possible food interaction.

Six animals were selected in order to obtain a complete pharmacokinetic profile.

The animals were administered individually by subcutaneous route in the inter scapular area. The areas were disinfected with an alcoholic solution (Diolina®, Braun-Dexon). The theoretical dose level of GLP-1(7-36)-NH₂ was 15 mg (approximately 150 μl of formulation per dog) in pre-filled individual 0.3-ml Terumo Myjector syringes with 12×0.33 mm Unimed needles.

The blood samples of about 2.0 ml were obtained, through the cephalic veins, before injection (time 0) and at several time points after administration along 35 days.

Blood was thereafter placed into pre-chilled 4-ml polyethylene tubes containing a 15% EDTA-K₃ aqueous solution (12 μl per ml of blood) as anticoagulant, Preservatives were added, Trasylol® (50 KIU or 5 μl per ml of blood) and DPP-IV inhibitor (10 μl per ml of blood). The blood samples remained in a cold water bath before centrifugation (1600 g for 20 min at 4° C. in the Sigma K4-15 centrifuge). Finally, the plasma was decanted into polypropylene cryotubes and moved rapidly in a −80° C. freezer before analysis.

The GLP-1(7-36)-NH₂ concentration was determined in plasma samples after a solid phase extraction of 0.3 ml of dog plasma and followed by solid phase extraction coupled to LC-MS/MS (API4000), using a GLP-1 analogue as internal standard. This method was carried out for measurement of GLP-1(7-36)-NH₂ dog plasma concentrations ranging from 0.25 ng/ml to 25 ng/ml.

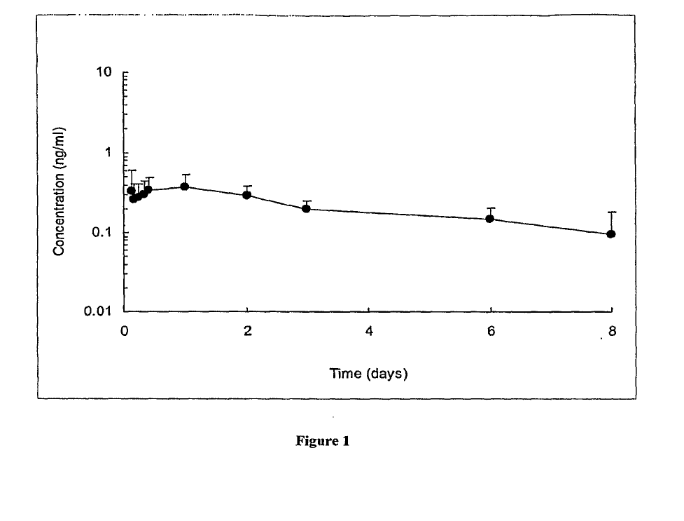

The peptide plasma profile obtained after single subcutaneous administration to dogs of the composition disclosed in example at the dose level of D=15 mg peptide (906.1 μg/kg), is shown in FIG. 1.

E.6. Additional Pharmacokinetic Study A

The same composition disclosed in E.5.1 is kept at 5° C. during at least 1 week and tested as described in previous example (E.5.2).

E.7. Additional Pharmacokinetic Study B

The same composition disclosed in E.5.1, is tested, at a dose higher than 15 mg peptide.

E.8. Additional Pharmacokinetic Study C

A similar composition, as prepared in E.5.1, is tested, at a peptide concentration lower than 100 mg/g

E.9. Additional Pharmacokinetic Study D

A similar composition, as prepared in E.5.1, is tested having a Peptide/Zn molar ratio higher than 1.5:1

E.10. Additional Pharmacokinetic Study E

A similar composition, as prepared in E.5.1, is tested, having a Peptide/Zn molar ratio higher than 1.5:1 and a peptide concentration lower than 100 mg/g

1. A pharmaceutical composition comprising a clear solution or an aqueous mixture, a suspension or a semisolid pharmaceutical composition of (a) at least one peptide compound having an aqueous solubility greater than 1 mg/mL at room temperature and having a pH from 3.0 to 8.0, and preferably a pH from 4.0 to 6.0 which is selected from the group consisting of hGLP-1(7-36)-NH₂ and analogs and derivatives thereof, hGLP-1(7-37)-OH and analogs and derivatives thereof, exendin-4 and analogs and derivatives thereof, 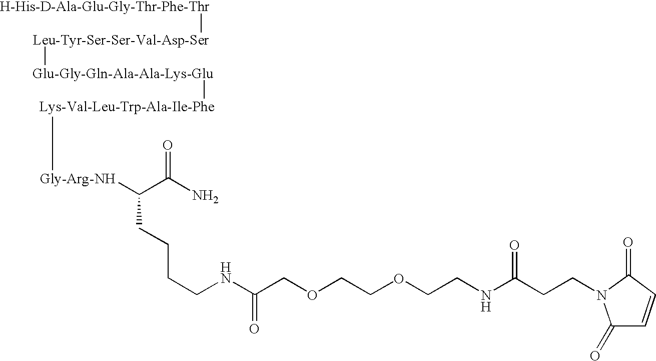
and analogs and derivatives thereof, 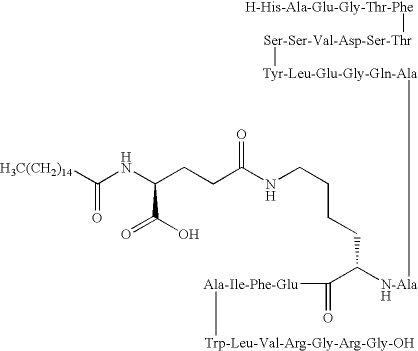
and analogs and derivatives thereof and H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-Lys-Lys-Lys-Lys-Lys-Lys-NH₂ and analogs and derivatives thereof; (b) a divalent metal ion; and
(c) a solvent
provided that less than 95%±5% of the said peptide compound is dissolved by said solvent.

2. A composition according to claim 1, wherein said divalent metal ion is zinc.

3. A composition according to claim 1 wherein said solvent is water.

4. A composition according to claim 1 wherein said solvent is a non-aqueous medium.

5. A composition according to claim 3 which further comprises a non-aqueous medium.

6. A composition according to claim 1 wherein said peptide compound is present in a concentration of about 0.00001-500 mg/mL, preferable about 0.0001-10 mg/mL.

7. A composition according to claim 2 wherein said zinc is present in a concentration from 0.0005 mg/mL to 50 mg/mL.

8. A composition according to claim 1 further comprising a preservative.

9. A composition according to claim 8 wherein said preservative is selected from the group consisting of m-cresol, phenol, benzyl alcohol and methyl paraben.

10. A composition according to claim 9 wherein said preservative is present in a concentration from 0.01 mg/mL to 50 mg/mL.

11. A composition according to claim 1 further comprising an isotonic agent.

12. A composition according to claim 1 wherein said isotonic agent is present in a concentration from 0.01 mg/mL to 50 mg/mL.

13. A composition according to claim 1 further comprising a stabilizer.

14. A composition according to claim 13 wherein said stabilizer is selected from the group consisting of imidazole, arginine and histidine.

15. A composition according to claim 1 further comprising a surfactant.

16. A composition according to claim 1 further comprising a chelating agent.

17. A composition according to claim 1 further comprising a buffer.

18. A composition according to claim 17 wherein said buffer is selected from the group consisting of Tris, ammonium acetate, sodium acetate, glycine, aspartic acid and Bis-Tris.

19. A composition according to claim 1 further comprising a basic polypeptide.

20. A composition according to claim 19 wherein said basic polypeptide is selected from the group consisting of polylysine, polyarginine, polyornithine, protamine, putrescine, spermine, spermidine and histone.

21. A composition according to claim 1 further comprising alcohol or mono- or di-saccharide.

22. A composition according to claim 2 wherein said alcohol or mono- or di-saccharide is selected from the group consisting of methanol, ethanol, propanol, glycerol, trehalose, mannitol, glucose, erythrose, ribose, galactose, fructose, maltose, sucrose and lactose.

23. A composition according to claim 1 further comprising ammonium sulfate.

24. A pharmaceutical composition comprising an effective amount of a compound according to claim 1 or a pharmaceutically acceptable salt thereof and a pharmaceutically acceptable carrier or diluent.

25. A method of eliciting an agonist effect from a GLP-1 receptor in a subject in need thereof which comprises administering to said subject an effective amount of a compound according to claim 1 or a pharmaceutically acceptable salt thereof.

26. A method of treating a disease selected from the group consisting of Type I diabetes, Type II diabetes, obesity, glucagonomas, secretory disorders of the airway, metabolic disorder, arthritis, osteoporosis, central nervous system disease, restenosis and neurodegenerative disease, in a subject in need thereof which comprises administering to said subject an effective amount of a compound according to claim 1 or a pharmaceutically acceptable salt thereof.

27. A method according to claim 26 wherein said disease is Type I diabetes or Type II diabetes.
